(19) United States
(12) Patent Application Publication
(10) Pub. No.: US 2012/0302440 A1
(43) Pub. Date: Nov. 29, 2012

(54) ABSORBENT ARTICLE COMPRISING A COMPOSITE MATERIAL

(75) Inventors: Hans Theliander, Goteborg (SE); Fredrik Wernersson, Vastra Frolunda (SE); Harald Brelid, Goteborg (SE); Kristoffer Lund, Goteborg (SE); Charlotta Hanson, Goteborg (SE); Ingrid Gustafson, Asa (SE); Torgny Falk, Hising-Backa (SE)

(73) Assignee: SCA HYGIENE PRODUCTS AB, Goteborg (SE)

(21) Appl. No.: 13/521,844

(22) PCT Filed: Jan. 18, 2011
(86) PCT No.: PCT/SE2011/050047

§ 371 Date: Jul. 12, 2012

(30) Foreign Application Priority Data
Jan. 19, 2010 (SE) PCT/SE2010/050046

(51) Int. Cl. B01J 20/24; B01J 20/28; A61F 13/534

(52) U.S. Cl. 502/404; 604/376

(57) ABSTRACT

An absorbent article including a freeze dried composite material. The freeze dried composite material includes cellulosic pulp (e.g. CTMP) and an absorbent material. The absorbent material includes microfibrillated cellulose (MFC) with a specified content of carboxylate groups.

### CROSS-REFERENCE TO RELATED APPLICATIONS

This application is a §371 National Stage Application of PCT International Application No. PCT/SE2011/050047 filed Jan. 18, 2011, which claims priority to PCT International Application No. PCT/SE2010/050046 filed Jan. 19, 2010, both of which are incorporated herein in their entirety.

### TECHNICAL FIELD

The present disclosure relates to an absorbent article including a composite material. The composite material includes an absorbent material in the form of an absorbent porous foam. The composite material is derived from a renewable cellulose source.

### BACKGROUND

Advances in absorbent article technology have stimulated the search for absorbent materials with desirable properties, such as high absorption, high storage capacity and high mechanical strength.

Absorbent articles, such as diapers, pantyliners, incontinence guards, sanitary napkins and the like typically include a superabsorbent material distributed within a fibrous matrix. Superabsorbent polymers (SAPs) are lightly crosslinked hydrophilic polymers having the ability to absorb and retain large amounts of liquid relative to their own mass. Hence, SAPs are widely used in absorbent articles to increase their absorbent capacity.

A variety of SAP materials have been described for use in absorbent articles, including both synthetic and natural SAPs. Natural materials, such as pectin, starch and cellulose-based materials typically suffer from poor absorption properties and low mechanical strength, and have thus not gained wide use in absorbent articles. On the other hand, synthetic materials, such as polyacrylic acid/polyacrylate SAPs are mainly derived from non-renewable raw materials such as fossil based oil, and are generally not recognized as environmentally sound.

The non-renewable nature of polyacrylate-based SAPs is an increasing concern in society, and it is desirable to find a biodegradable and renewable material having absorption characteristics similar to synthetic SAP materials.

Environmental concerns have led to several attempts directed to the use of cellulose, which is a biodegradable and renewable resource. For example, US 2003/0045707 relates to a superabsorbent polymer derived from a cellulosic, lignocellulosic, or polysaccharide material, wherein the polymer is preferably sulfated to increase its water swellability. WO 97/21733 discloses a water-swellable, water-insoluble sulfonated cellulose having an average degree of sulfonic group substitution from about 0.2 to about 0.5.

In recent years, microfibrillated cellulose (MFC) has attracted considerable attention in various applications. This is particularly attributed to its high mechanical performance and stability.

For example, EP 0210 570 discloses an absorbent retentive pulp produced by subjecting a microfibrillated pulp slurry to pore generation particles and to cross-linking with a cross-linking agent.

Similar approaches are disclosed in U.S. Pat. No. 4,474,949 and EP 0 209 884, wherein an absorbent retentive pulp is provided by mechanically treating cellulosic fibres into microfibrillar form and subjecting the pulp to freeze-drying.

In view of the growing interest in replacing traditional polyacrylate based SAP materials with more environmentally sound alternatives, there is a need to provide alternative natural superabsorbent materials based on cellulose. Such materials should be mechanically stable, and exhibit improved absorption characteristics, making them suitable for incorporation into absorbent articles.

### SUMMARY

It is desired to fulfill the above mentioned need and to provide an absorbent article including a highly absorbent material which exhibits superior absorption characteristics and mechanical strength; the material being derived from a renewable cellulose-based source.

This can be achieved by an absorbent article according to the appended claims.

Thus, a first aspect relates to an absorbent article including a freeze dried composite material. The composite material includes cellulosic pulp and an absorbent material.

The absorbent material includes microfibrillated cellulose in the form of an absorbent porous foam. The microfibrillated cellulose (MFC) has a content of carboxylate groups of from 0.5 to 2.2 mmol/g of MFC.

The absorbent material exhibits unique stability and absorption properties and is environmentally sound.

The present inventors have found that by controlling the amount of charged groups; i.e. carboxylate groups in the cellulosic chains of the MFC, the characteristics of the porous foam structure are improved. The stability of the porous foam is enhanced and the absorption properties are improved.

Generally, the absorption capacity increases with the amount of charged groups. However, at high loading of charged groups on the MFC; i.e. above 2.2 mmol/g, the thin fibrils become more prone to degradation, which is undesirable. In contrast, if the content of charged groups is too low, the material tends to be less “foam like”, and a network of significantly larger freeze-dried cellulosic fibres is obtained. Such a material is less stable in the wet state and is brittle.

In the range of from 0.5 to 2.2 mmol/g of charged groups; i.e. carboxylate groups, the foam is characterized by a high content of fine pores capable of trapping large amounts of liquid, which in turn results in a good rate of absorption and wicking.

The microfibrillated cellulose (MFC) imparts a mechanical strength and stability to the foam by “locking” the foam structure and making it less prone to degradation.

The composite material includes the absorbent material described above and freeze dried cellulosic pulp. The present inventors have found that the performance of the absorbent material may be enhanced when present in the form of a composite. The porous foam imparts a stability to the cellulose pulp fiber network, and thus also to the composite material as such.

Less absorbent material is needed in the composite to provide a similar absorbent capacity, which is believed to be due to a positive synergetic effect between the components of the composite. If these two components are used separately, and become wetted, the absorbent material may not be able to withstand high compressive pressures, and the fiber network of the cellulosic pulp may fall apart. However, in the form of a composite, the absorbent material will act as a “glue” and form very resistant bonds between the fibers in the network. A relatively stiff material is thus obtained which is capable of resisting higher compressive forces. This will, in turn, have the consequence that the absorbent material will not be subjected to the high compressive forces and, thus a larger part of the material can be used for liquid storage.

The composite material is substantially made from renewable sources; i.e. cellulose based materials, and thus presents an environmentally sound alternative for use in hygiene products instead of traditional fibrous structures including superabsorbent polymers based on fossil oil.

The composite material may include at least 5% by weight of the absorbent material. In a particular embodiment, the composite material includes from 10 to 50% by weight absorbent material, e.g. 10 to 30% by weight. The present inventors have found that even small amounts of absorbent material provide for good absorption and material compression properties of the composite. Since cellulosic pulp is typically an inexpensive material, the composite material is also advantageous from an economical point of view.

In a particular embodiment, the pulp is chemithermomechanical pulp (CTMP). A composite material including CTMP has a high mechanical strength and a high wet bulk.

In a certain embodiment, the content of charged groups in the microfibrillated cellulose is from 0.8 to 1.8 mmol/g of MFC. This results in an enhanced foam stability and an improved absorption.

The microfibrillated cellulose can have a content of carbonyl groups of at least 0.2 mmol/g of MFC, preferably of at least 0.5 mmol/g. The carbonyl groups enhance the stability of both the absorbent material as such, but also of the composite material. These groups may form interfibrillar covalent bonds within the porous foam structure as well as between the fibers of the cellulose fiber network. A highly rigid, mechanically stable structure is thereby obtained.

The absorbent material has a BET surface area of at least 24 m2/g, preferably at least 30 m2/g. This allows for a large surface area to become accessible to a liquid and the fineness of the solid phase of the foam is increased. Consequently, this influences the absorption properties of the material. For example, the capillarity; i.e. the capillary suction, is improved, which may provide good liquid retention, and may also allow for some wicking of the fluid to occur within the foam structure.

The absorbent material has a wet bulk of at least 10 cm3/g at 5 kPa, preferably at least 15 cm3/g at 5 kPa. Accordingly, the absorbent material; i.e. the absorbent porous foam is mechanically stable under load; i.e. it has the ability to retain large amounts of liquid and does not “collapse” upon exposure to excess liquid.

Furthermore, the absorbent material has a free swell capacity (FSC) value of at least 45 g/g. This demonstrates good absorption capacity of the absorbent material.

In addition to the good liquid absorption properties, the absorbent article also displays good liquid retention capacity. The absorbent material; i.e. the porous foam has a retention capacity (CRC) as determined by the Centrifuge Retention Capacity Test of at least 8 g/g, preferably at least 12 g/g. Hence, the foam has the ability to firmly trap and retain liquid within the pores and cavities of the foam.

The composite material may be obtainable by:
(a) oxidizing a first cellulosic pulp to obtain a content of carboxylate groups of from 0.5 to 2.2 mmol/g of pulp,
(b) disintegrating said first cellulosic pulp into microfibrillated cellulose,
(c) mixing the microfibrillated cellulose of step b) with a second cellulosic pulp, and
(d) freeze-drying said mixture of microfibrillated cellulose and said second cellulosic pulp.

A freeze dried composite material is thereby formed, wherein the absorbent foam material is distributed within the pulp fiber structure. A fine pore structure is formed in the space between the larger fibers. The composite formed by the method above is mechanically stable and does not require any additional crosslinking agents to keep the material together. Such crosslinking agents are typically required to keep the structure together in ordinary microfibrillar materials.

The microfibrillated cellulose and the second cellulosic pulp are typically mixed in the wet state.

In certain embodiments, the oxidation step (a) is performed in the presence of 2,2,6,6-tetramethylpiperidine-1-oxyl (TEMPO). This oxidation method allows for a selective and controlled oxidation, mainly directed towards the hydroxyl groups at carbon 6 of the cellulose chains. It also allows for the formation of carbonyl groups, which as mentioned above, contribute to the stability of the absorbent foam.

The absorbent article typically includes a liquid permeable topsheet, a backsheet and an absorbent body enclosed between the liquid-permeable topsheet and the backsheet. The composite material including the absorbent material is present in the absorbent body.

Since the porous absorbent foam has multifunctional absorption properties with respect to liquid absorption, acquisition, and storage capacity, the composite material may simultaneously fulfill the functions of a liquid acquisition layer, liquid distribution layer and liquid storage layer.

The absorbent body or at least one layer thereof may include fractions of the composite material mixed with a second absorbent material. This arrangement may improve the liquid spreading within the absorbent body.

These and other aspects will be apparent from and elucidated with reference to the embodiment(s) described hereinafter.

### BRIEF DESCRIPTION OF THE DRAWINGS

FIG. 1 illustrates the SEM structure of an absorbent porous foam according to an embodiment of the present invention (1a) compared to a reference material (1b). FIG. 1c illustrates the SEM structure of the composite material of an embodiment of the invention.

FIG. 2a illustrates the wet bulk of the absorbent material of an embodiment of the present invention compared to a reference material.

FIG. 2b illustrates the wet bulk (5.2 kPa) of the freeze dried composite materials formed from different types of cellulosic pulp fibres and with different amounts of such fibres compared to the theoretical values for each material.

FIG. 3 illustrates the free swell capacity of the absorbent material of an embodiment of the present invention compared to a reference material.

FIG. 4a illustrates the centrifuge retention capacity of the absorbent material of an embodiment of the present invention compared to a reference material.

FIG. 4b illustrates the centrifuge retention capacity of the absorbent material when it has been combined with cellulosic fibres in a freeze dried composite material.

FIG. 5a is a schematic overview of a process used to manufacture the absorbent material of an embodiment of the present invention.

FIG. 5b schematically illustrates a process by which the composite material of an embodiment of the present invention may be produced.

FIG. 6 illustrates the total cumulative volume of liquid with respect to the pore radius.

FIG. 7 illustrates an absorbent article according to an embodiment of the present invention.

FIG. 8 illustrates an absorbent article according to an embodiment of the present invention in transverse cross-sectional view through the mid-point of the article.

### DETAILED DESCRIPTION OF PREFERRED EMBODIMENTS

The present disclosure relates to an absorbent article including an absorbent material. The absorbent material includes freeze-dried microfibrillated cellulose in the form of an absorbent porous foam. The freeze-dried microfibrillated cellulose (MFC) includes charged groups in an amount of from 0.5 to 2.2 mmol/g of MFC.

The absorbent material is included within a freeze dried composite material, which also includes cellulosic pulp.

The term “absorbent article” includes any type of absorbent hygiene article, e.g. diapers, incontinence care articles, feminine hygiene articles such as sanitary napkins, and the like. It may also include any type of tissue-towel paper products for facial tissue, toilet tissue, absorbent paper towels and handkerchiefs.

The term “freeze dried composite material” means a freeze dried structure made up of at least two distinct components: absorbent material in the form of a porous foam, and cellulosic pulp. These components are interconnected by stable interfibrillar bonds and remain separate and distinct on a microscopic level in the composite. The components of the composite are typically mixed in the wet state. Other components may also be present in the composite.

As used herein, the term “porous” refers to a material including pores and which admits the passage of gas or liquid through these pores.

The term “foam” refers to a material formed by trapping gas bubbles in a liquid or solid. A “foam”, within the meaning of embodiments of the present invention, also refers to a structure produced by trapping water domains in a solid and subsequently vaporizing the water using a freeze-drying process.

The absorbent material of embodiments of the present invention is an “absorbent porous foam” which is a solid foam composed of a continuous phase based on microfibrillated cellulose which surrounds pores that are connected to each other and form an interconnected porous system.

The term “microfibrillated cellulose” or “MFC”, as used herein, refers to small diameter, high length-to-diameter ratio substructures. The free and individual fibres typically have a diameter of from 5 nm to 300 nm, preferably of from 5 nm to 100 nm at all points along the fibre. The diameter may vary along its length. The microfibrillated cellulose may exist as free and individual fibrils and/or as free clusters of such fibrils.

The microfibrillated cellulose may be prepared from any source of cellulose including, without limitation, wood fibres, e.g. derived from hardwood and softwood, such as from chemical pulps, mechanical pulps, thermomechanical pulps, chemithermomechanical pulps, recycled fibres, seed fibres, leaf fibres, straw fibres or cellulosic fibres produced by bacteria.

The absorbent porous foam of the article is made from a renewable source (cellulose) and thus provides an environmentally sound alternative to conventional polyacrylate-based SAP materials. Due to its good liquid absorption, retention and storage properties, it is suitable for incorporation into any type of absorbent article.

The charged groups present in the foam; i.e. the microfibrillated cellulose, increase the osmotic pressure such that liquid is efficiently and rapidly absorbed into the foam. This, in turn, may affect the capillary force needed to retain the liquid within the foam structure. Accordingly, the absorbent material allows for improved absorbing, liquid spreading and liquid storage properties of the absorbent article.

As used herein, the term “charged group” refers to any negatively charged entity. Typically, the charged groups are carboxylate groups. Such carboxylate groups may be generated by oxidation of the cellulose chain, preferably at carbon 6; i.e. the carbon including a free hydroxyl group (marked with * below).

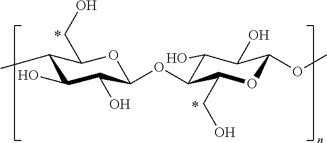

The content of charged groups, e.g. carboxylate groups is defined as the molar amount per gram of microfibrillated cellulose or per gram of pulp, and is expressed in mmol/g.

The amount of charged groups in a range of 0.5 to 2.2 mmol/g of MFC has proved to be advantageous in terms of providing desirable absorption properties.

In this range, the absorbent material is characterized by a porous foam including a high content of fine pores capable of trapping a large amount of liquid, which in turn results in an improved rate of absorption and an enhanced wicking ability; i.e. the ability of the foam to distribute liquid within the foam.

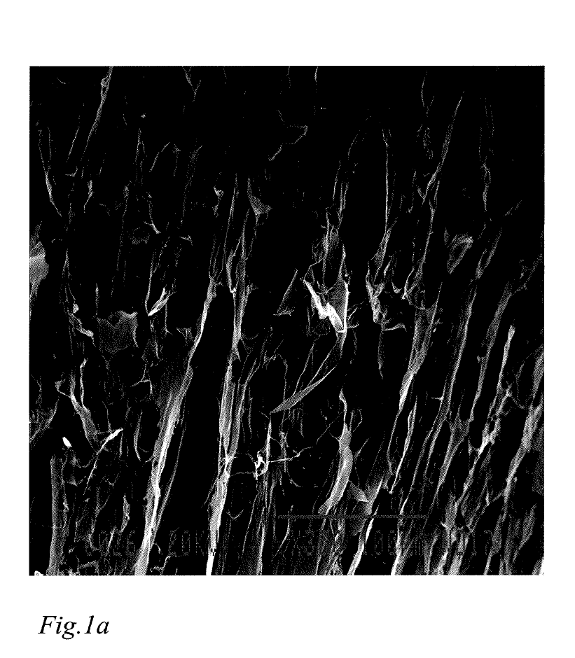

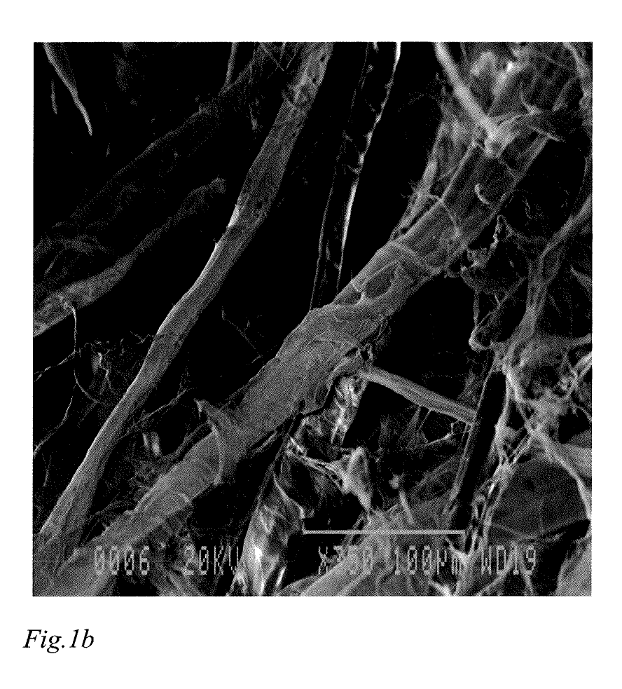

However the content of charged groups should not exceed 2.2 mmol/g since an excess of charged groups may make the MFC more prone to degradation, which is undesirable. In contrast, if the content of charged groups is too low, e.g. below 0.5 mmol/g, the material tends to lose its foam characteristics and typically contains larger fibres with a considerable amount of external fibrillation (see FIG. 1b).

The freeze-dried microfibrillated cellulose imparts a mechanical strength and stability to the porous foam material and has the ability to “lock” the foam structure. The improved stability of the absorbent porous foam is believed to be due to particularly strong hydrogen bonds between the thin and flexible fibrils of the microfibrillated cellulose, which strengthen the foam structure. In addition, the stability of the foam may be attributed to the presence of carbonyl groups in the microfibrillated cellulose. These groups may provide crosslinks between the MFC fibrils, which serve to enhance the stability of the material by forming interfibrillar covalent bonds within the absorbent porous foam.

The absorbent porous foam contains pores and cavities that are connected to each other to form a fine interconnected network. Such a foam is stable both in dry and wet conditions, and does not fall apart under pressure.

The composite material includes the absorbent material described above, referred to herein as “the absorbent material”, and cellulosic pulp. In particular embodiments, these two components have been mixed in the wet state, and thereafter been freeze dried. Very strong interfibrillar bonds are thereby formed between the pulp fibers and the absorbent material. The absorbent material; i.e. the absorbent porous foam is distributed between the larger freeze dried cellulosic fibers and acts as a “glue” to keep the material together.

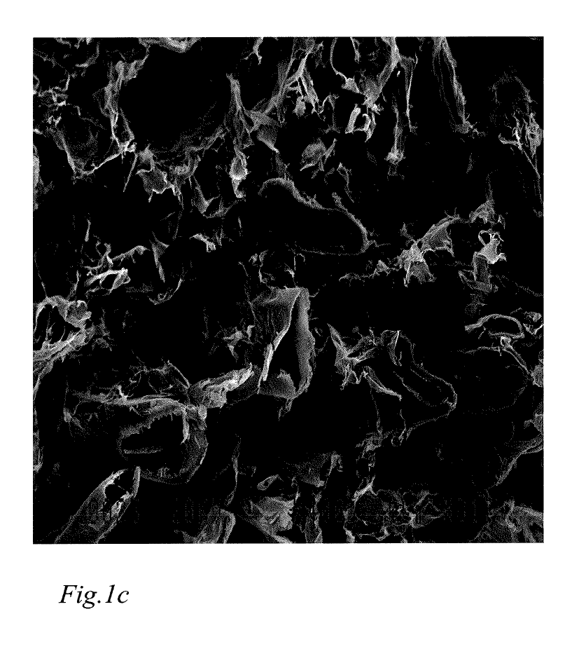

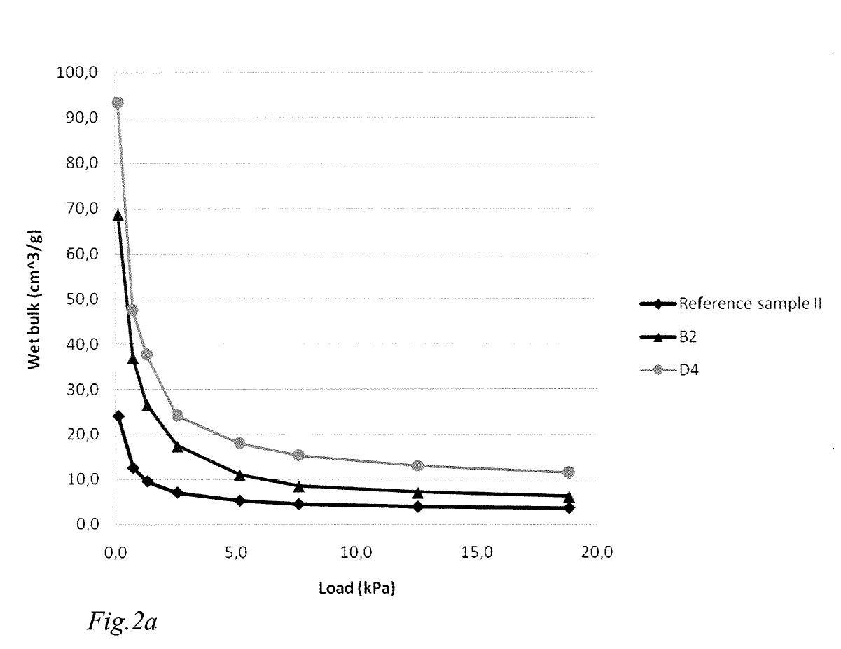

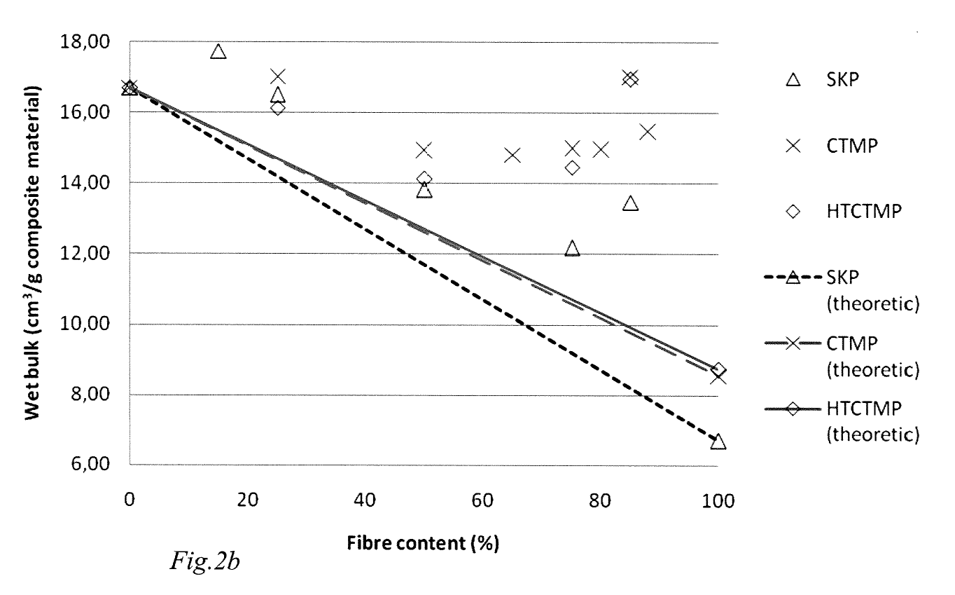

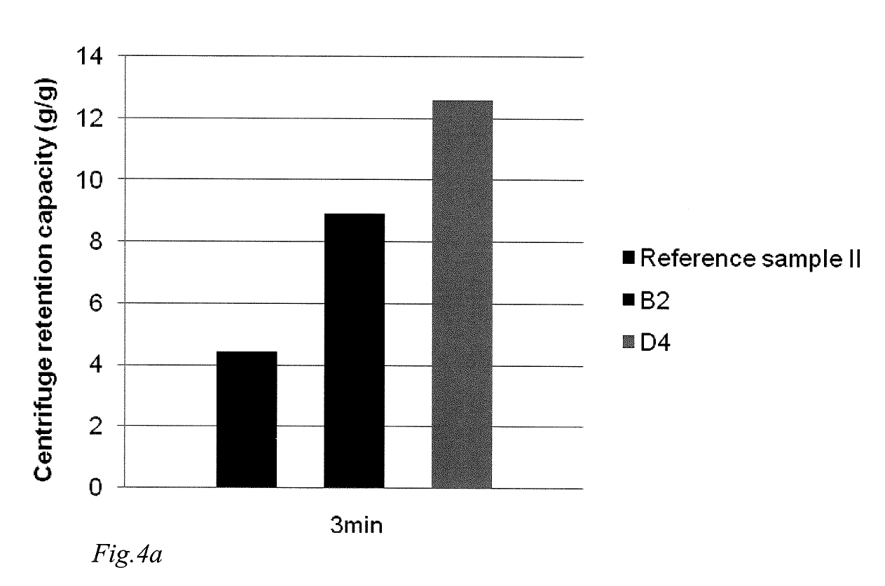

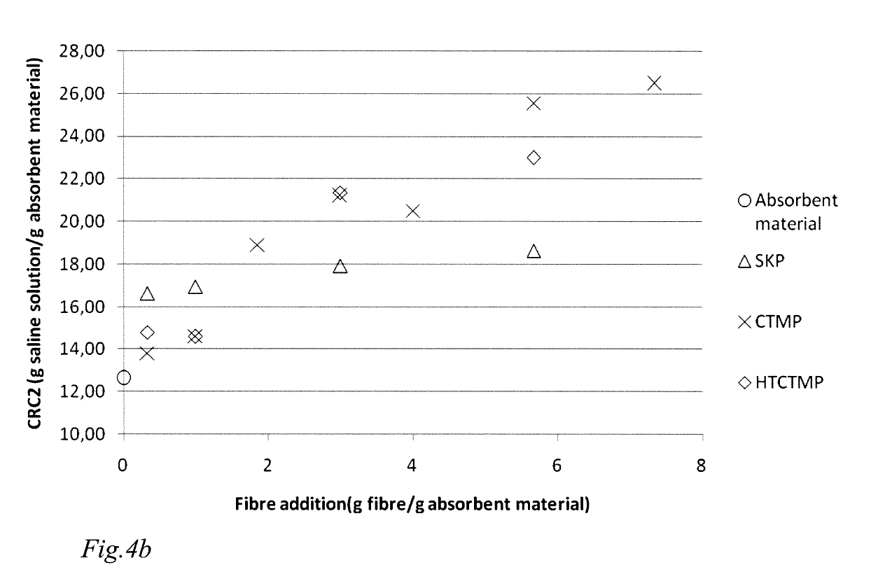

The inventors have found that the composite material has a very good absorption capacity under pressure, as illustrated by a high wet bulk in FIG. 2b. Furthermore, the composite material gives a more efficient utilization of the retention capacity of the absorbent material compared to the absorbent material as such (see FIG. 4b).

It is believed that the fibers of the cellulosic pulp, when associated with the absorbent material, improve the mechanical properties of the composite material such that the structure is able to withstand higher mechanical stresses. The material can be compressed to high densities, and yet expand when wetted.

The presence of the cellulosic pulp fibers within the freeze dried composite may also improve the liquid distributing capacity of the material.

The composite material is unique in the sense that it is a substantially wood based material. The absorbent properties are, however, similar to absorbent structures including superabsorbent polymers based on fossil oil.

The composite material may include at least 5% by weight of the absorbent material. In a certain embodiment, the composite material includes of from 10 to 50% by weight absorbent material, e.g. 10 to 30% by weight. Even such small amounts of absorbent material are enough to provide good absorption properties. As mentioned above, the absorbent material acts as a “glue” in the fiber intersections and forms very resistant bonds between the fibers in the network. A relatively stiff material is thus obtained which is capable of resisting high compressive forces. This will, in turn, have the consequence that the absorption material will not be subjected to the high compressive forces and, thus a larger part of the material can be used for liquid storage. A surprisingly high wet bulk has been observed, even when such small amounts of absorbent material are used.

An improved wet bulk has also been observed when the pulp is chemithermomechanical pulp (CTMP), e.g. high temperature chemithermo-mechanical pulp (HTCTMP). CTMP is an inexpensive material which generally has a low absorption capacity. It is thus surprising that the absorption properties are improved to such an extent for a composite comprising CTMP. This may be attributed to the fact that this pulp type is associated with long fiber lengths, a low fines content, strength, and stiffness of the fibers.

The microfibrillated cellulose of the absorbent material suitably has a content of charged groups; i.e. carboxylate groups of from 0.8 to 1.8 mmol/g of MFC. Particularly good absorption properties have been observed within this range. The foam structure includes many fine interconnected pores and is capable of absorbing over 180 times its own weight after dipping in water for 10 minutes. The liquid absorption is high (over 150 times its own weight) even after only one 1 minute, which demonstrates a remarkably rapid liquid intake (see table 6).

This is noteworthy and superior to conventional polyacrylate based SAP materials, which typically have a slow initial rate of absorption.

In particular embodiments, the microfibrillated cellulose has a content of carbonyl groups of at least 0.2 mmol/g of MFC, e.g. of at least 0.5 mmol/g of MFC. The carbonyl groups are able to form hemiacetal bonds and acetal bonds in reaction with hydroxyl groups present on the surface of the MFC and of the cellulosic pulp fibers. Hence, the stability of both the absorbent material as well as the composite material is enhanced.

The absorbent porous foam has a BET surface area of at least 24 m2/g, e.g. at least 28 m2/g, preferably at least 30 m2/g.

As used herein, the term “BET surface area” or “surface area” is a measure of the accessible area of the foam, to which a test liquid is exposed. Hence, it is a way of quantifying the total amount of solid surface provided by the absorbent porous foam.

When the foam has a large specific surface area, the absorption is improved and liquid may also be more efficiently retained within the foam structure. The BET surface area is determined by the accessible area (m2) per gram of foam material. A high BET surface area results in an improved rate of absorption and capillarity, allowing for an acceptable liquid retention and a desired wicking to occur within the foam structure.

As is illustrated in the SEM picture of FIG. 1a, the porous foam is characterized by very fine structures of microfibrillated cellulose in a sheet-like structure with large voids between them. This has the effect that, upon exposure to a liquid to be absorbed, a high quantity of surfaces is accessible. As a result, the absorption is enhanced.

In contrast, when the BET surface area is low, as illustrated in FIG. 1b, less surfaces are accessible within the foam material, and consequently, the absorption capacity decreases.

Another feature of the absorbent material is that it has a high wet bulk. As used herein, the term “wet bulk” refers to the volume of cubic centimetres per gram (dry basis) of the absorbent material under a load after the material has been saturated with deionized water. The wet bulk is correlated to the absorption under load. The test is designed to indicate the effectiveness of the absorbency in e.g. a diaper under the weight of a baby.

The absorbent material has a wet bulk of at least 10 cm3/g at 5 kPa, preferably at least 15 cm3/g at 5 kPa (see FIG. 2). Accordingly, the absorbent material has the ability to retain large amounts of liquid and does not “collapse” upon exposure to excess liquid. The foam may rapidly acquire and effectively distribute liquid to sites remote from insult.

The present inventors have surprisingly found that the wet bulk is increased when the absorbent material is included within a composite material. This is unexpected and proves the positive synergistic effect between the pulp fiber structure and the absorbent material in the freeze dried composite. Best results are achieved when CTMP or HTCTMP is used in the composite (see FIG. 2b).

The absorbent material; i.e. the absorbent porous foam has a free swell capacity (FSC) value of at least 45 g/g.

As used herein, the term “free swell capacity” or “FSC” means the absorbent capacity determined by soaking an absorbent material in a 0.9 percent aqueous sodium chloride solution during 30 minutes at room temperature, subsequently dripping off excess fluid, and weighing to determine the amount of fluid absorbed. The free swell capacity is expressed in terms of gram absorbed fluid per gram of dry weight of a sample.

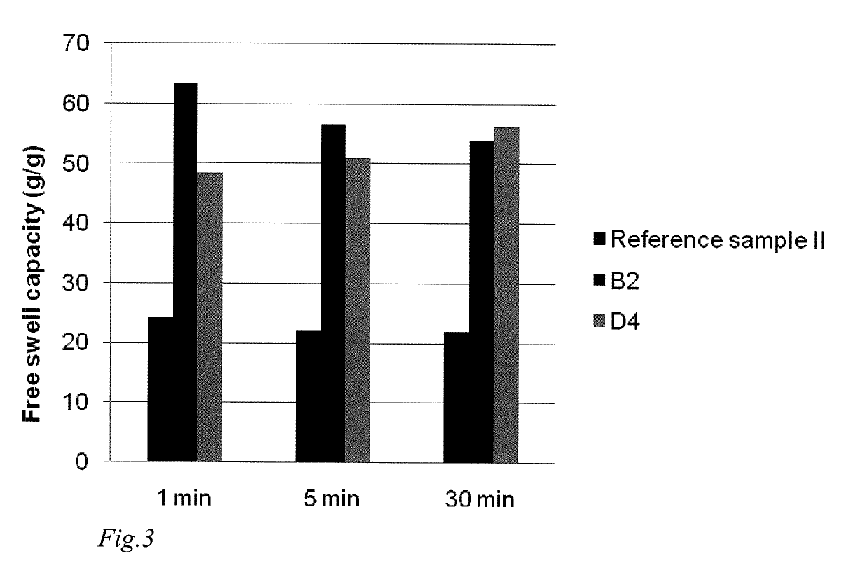

As is observed in FIG. 3, the free swell capacity of the absorbent material is very high even after 1 minute, and 5 minutes, respectively, and values up to 60 g/g have been observed. This demonstrates the enhanced absorption rate and rapid liquid uptake of the absorbent material. Such high FSC values are typically not observed for conventional polyacrylate based SAP materials, which, as mentioned hereinbefore generally exhibits a slow initial rate of absorption.

In addition to the improved liquid absorption properties, the absorbent material of the absorbent article also displays good liquid storage capacity as measured by the Centrifuge Retention Capacity (CRC) test.

As used herein, the term “centrifuge retention capacity” or “CRC” is a measure of the capacity of the foam to retain liquid within the absorbent material. The centrifuge retention capacity is measured by soaking an absorbent material in a 0.9 percent aqueous sodium chloride solution during 30 minutes at room temperature, and then centrifuging the material for 3 minutes to determine the amount of fluid retained.

The absorbent material has a retention capacity (CRC) as determined by the Centrifuge Retention Capacity Test of at least 8 g/g, e.g. at least 10 g/g, and preferably at least 12g/g. As compared to conventional pulp, the CRC is remarkably improved (see FIG. 4a).

The inventors have surprisingly found that the CRC value of the absorbent material increases with increased amounts of cellulosic pulp fibers in the composite. This means that the composite material gives a more efficient utilization of the retention capacity of the absorbent material compared to the absorbent material as such. As illustrated in FIG. 4b, very high CRC values were obtained at a high concentration of pulp fibers, which indicates a better preservation of the porous structure (less structure collapses) at high fiber additions.

The absorbent porous foam suitably has a total cumulative volume of more than 5 mm3/mg, preferably more than 10 mm3/mg, at a corresponding pore radius of 2 μm. The absorbent porous foam may have a total cumulative volume or more than 20 mm3/mg, preferably more than 40 mm3/mg, in an interval of corresponding pore radii from 10 μm to 50 μm. Such a foam is useful as it has larger voids that may give better liquid transportation and smaller voids that have better retention properties.

The absorbent material; i.e. the absorbent porous foam of the article of an embodiment of the present invention may be obtained by:
(a) oxidizing a cellulosic pulp to provide a content of carboxylate groups of from 0.5 to 2.2 mmol/g of pulp,
(b) disintegrating the cellulosic pulp into microfibrillated cellulose, and
(c) freeze-drying the microfibrillated cellulose.

The content of carboxylate groups may be measured and determined by any known method, e.g. sorption by methylene blue. This method is further described in P Fardim, B Holmbom, J Karhu, Nordic Pulp and Paper Research Journal 2002, 17:3, 346-351, which is incorporated herein by reference.

Step (a) may be achieved by controlled oxidation using any type of oxidizing agent; i.e. an agent which oxidizes the hydroxyl groups on the glucose units of the cellulose chains. For example, sodium periodate or nitrogen dioxide may be used. Alternatively, the cellulosic pulp may be subjected to carboxymethylation, wherein monochloric acetic acid reacts with the hydroxyl groups of the cellulosic chains of the pulp to generate charged groups.

The oxidation may also be performed by a free radical reaction. Such a reaction is initiated by the reaction with a catalytic agent to generate a free radical. The oxidizing agent in a free radical reaction is a carrier of the free radical, e.g. hypohalites, such as hypofluorites, hypochlorites, hypobromites, and hypoiodites, preferably hypochlorites such as sodium hypochlorite (NaOCl), potassium hypochlorite (KOCl), lithium hypochlorite (LiOCl), or calcium hypochlorite (Ca(OCl)2). The list of examples of oxidizing agents is not exhaustive. The catalytic agent may be a peroxide or an organic nitroxyl compound, such as 2,2,6,6-tetramethylpiperidin-1-oxyl (TEMPO), 2,2,5,5,-tetramethylpyrrolidine-N-oxyl (PROXYL), 4-hydroxy-2,2,6,6-tetramethylpiperidin-1-oxyl, and 4-acetamido-2,2,6,6-tetramethylpiperidin-1-oxyl, and derivatives thereof. These catalytic agents react with selectivity on carbon-6 of the glucose entity of the cellulose molecule.

Prior to oxidation (step a), the cellulose pulp may be refined, for example by additional steps of mechanical treatment before the oxidation step (a). This may be advantageous when considering optimisation of energy demand of the process.

The presence of charged groups allows the cellulosic pulp to be more easily disintegrated into microfibrillated cellulose (step b).

The step of disintegrating the cellulosic pulp is typically achieved by homogenizing pulp into finer structures; i.e. microfibrillated cellulose, e.g. by means of an ultrasonic homogenizer. The degree of homogenization required depends on the amount of charged groups imparted to the pulp. For example, if the content of charged groups is high, the homogenization time may be as low as one or a few minutes. In contrast, if the content of charged groups is lower, a homogenization time of above 10 minutes may be required. The microfibrillated cellulose may be present as individual MFC fibrils or clusters thereof.

The material resulting from the mechanical treatment in step (b) has a gel-like character.

By freeze-drying the microfibrillated cellulose, an absorbent porous foam including many interconnected pores and thin MFC fibrils and clusters thereof is obtained. Other drying techniques, such as air-drying do not lead to such foam characteristics.

The charged groups imparted to the cellulosic pulp in step (a) remain even after the pulp has been subjected to mechanical treatment (step b) and freeze-drying (step c); i.e. the freeze-dried microfibrillated cellulose includes essentially the same amount of charged groups as that of the pulp of step (a).

The composite material of embodiments of the invention may be obtained by:
(a) oxidizing a first cellulosic pulp to obtain a content of carboxylate groups of from 0.5 to 2.2 mmol/g of pulp,
(b) disintegrating said first cellulosic pulp into microfibrillated cellulose,
(c) mixing the microfibrillated cellulose of step b) with a second cellulosic pulp, and
(d) freeze-drying said mixture of microfibrillated cellulose and second cellulosic pulp.

Steps (a) and (b) may be performed as described above. In step (c), the microfibrillated cellulose is mixed with a second cellulosic pulp, and thereafter the mixture is freeze dried.

The second cellulosic pulp may be any type of cellulose based pulp, such as chemical, mechanical or thermal mechanical pulp. In particular embodiments, the second cellulosic pulp is chemithermomechanical pulp (CTMP), e.g. high temperature chemithermomechanical pulp (HTCTMP).

FIG. 1c illustrates a composite material formed according to the method above. CTMP fibers are embedded in a matrix of absorbent material. The absorbent material has a large surface area with small pores. The pores of the absorbent material remain preserved even when exposed to high pressures.

In particular embodiments, step (a) is performed in the presence of 2,2,6,6-tetramethylpiperidine-1-oxyl (TEMPO). TEMPO is a preferred catalytic agent as it allows for a selective and controlled oxidation. The hydroxyl groups at carbon 6 in the cellulose chains of the cellulosic pulp can preferably be oxidized to charged carboxyl groups in an amount of from 0.5 to 2.2 mmol/g of pulp. This catalytic agent is stable during the reaction and can also be recovered and recycled into the process which is an important aspect from both an economical and an environmental perspective. Furthermore, this oxidation method does not cause any extensive deterioration of the cellulose chains of the pulp, which may be the case with other oxidation methods.

Co-catalysts may also be added, e.g. alkali metal bromides such as sodium bromide (NaBr), potassium bromide (KBr), and lithium bromide (LiBr).

During oxidation in the presence of TEMPO, carbonyl groups are generated, and these groups may enhance the stability by forming interfibrillar covalent bonds within the absorbent porous foam. Thereby, covalent crosslinks between the MFC fibrils may be formed, which are important for the preservation of the fibril network in the wet state. In ordinary microfibrillar materials, crosslinking agents are typically required to keep the material together. However, due to the strong interfibrillar bonds created within the absorbent porous foam material, crosslinking agents are not required. The first cellulosic pulp can preferably be oxidized to obtain a content of carbonyl groups of at least 0.2 mmol/g MFC, e.g. at least 0.5 mmol/g.

The interfibrillar covalent bonds contribute to the mechanical strength of the foam and the composite as such.

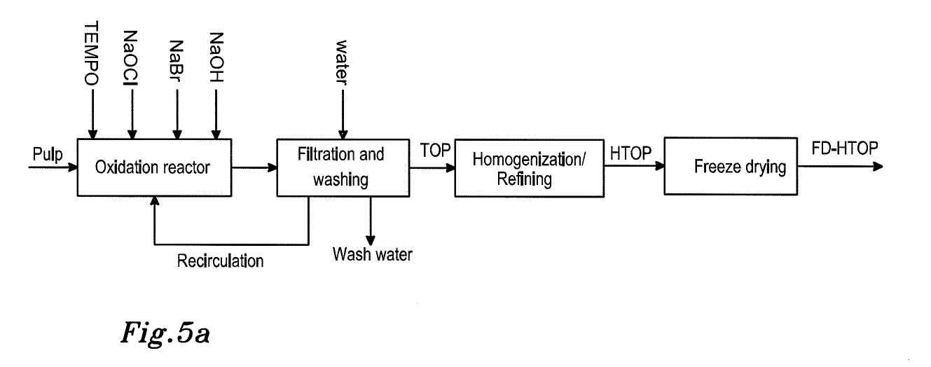

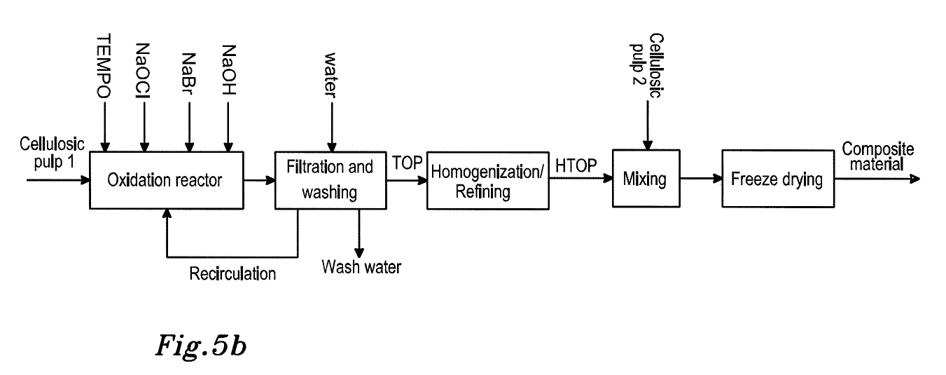

FIG. 5a schematically illustrates a process by which the absorbent foam may be produced.

In the first step (step a), the reaction is started by the addition of an oxidation agent, e.g. sodium hypochlorite (NaOCl), which may be added in amount of about 1.5 to 7.0, e.g. 2.0 to 6.0 mmol/g pulp. NaOCl reacts with e.g. sodium bromide (NaBr) to generate hypobromite. The amount of NaBr may e.g. be 0.2-8 mmol. Hypobromite subsequently oxidizes 2,2,6,6-tetramethylpiperidin-1-yloxy (TEMPO), which facilitates the oxidation of hydroxyl groups at carbon 6 of the cellulose chains. The amount of TEMPO may be e.g. 0.01-0.5 mmol/g. In this step, negatively charged carboxyl groups as well as carbonyl groups are generated, which contribute to the absorption properties and the mechanical stability of the foam, respectively.

An alkaline compound, such as sodium hydroxide (NaOH) is added to keep the pH between 8.5 and 10.5 such that the cellulosic fibres do not extensively degrade or deteriorate during the reaction.

The process may further include a washing step, wherein the pulp is washed and filtered to recycle the catalytic and oxidizing agents; i.e. TEMPO, NaBr etc., and to separate undesired dissolved fibre components and material.

After the oxidation, the pulp is referred to as a TEMPO oxidized pulp (TOP).

The TOP is then subjected to mechanical treatment (step b); i.e. homogenization to disintegrate the cellulosic pulp into microfibrillated cellulose. The material obtained is referred to as homogenized TEMPO oxidized pulp (HTOP). The step of disintegrating may be achieved by any method, wherein forces are applied to the cellulose pulp to disintegrate the fibres of the pulp, e.g. mechanical beating.

The HTOP is subsequently freeze-dried to generate a porous foam including freeze-dried microfibrillated cellulose including charged groups in an amount of from 0.5 to 2.2 mmol/g of MFC (step c).

FIG. 5b schematically illustrates the process by which the composite material may be formed. It includes the same steps as that of FIG. 5a, but further comprises a mixing step, wherein the homogenized TEMPO oxidized pulp (HTOP) is mixed with a second cellulosic pulp. The mixture is thereafter freeze dried. The two materials can preferably be mixed in the wet state prior to freeze drying to allow the formation of stable chemical bonds between the pulp fibers and the absorbent material.

The composite material and the absorbent porous foam can preferably be free from any traditional superabsorbent polymers (SAP). It is however conceivable, within the scope of the invention to add SAP to the composite or to the foam structure.

“Superabsorbent polymers” are water-swellable, water-insoluble organic or inorganic materials capable of absorbing at least about 20 times their own weight and in an aqueous solution containing 0.9 weight percent (wt %) of sodium chloride. Any type of superabsorbent polymer (SAP) known to those skilled in the art may be incorporated within the foam.

Additional components such as viscosity control agents and surfactants may also be added to improve the stability of the foam.

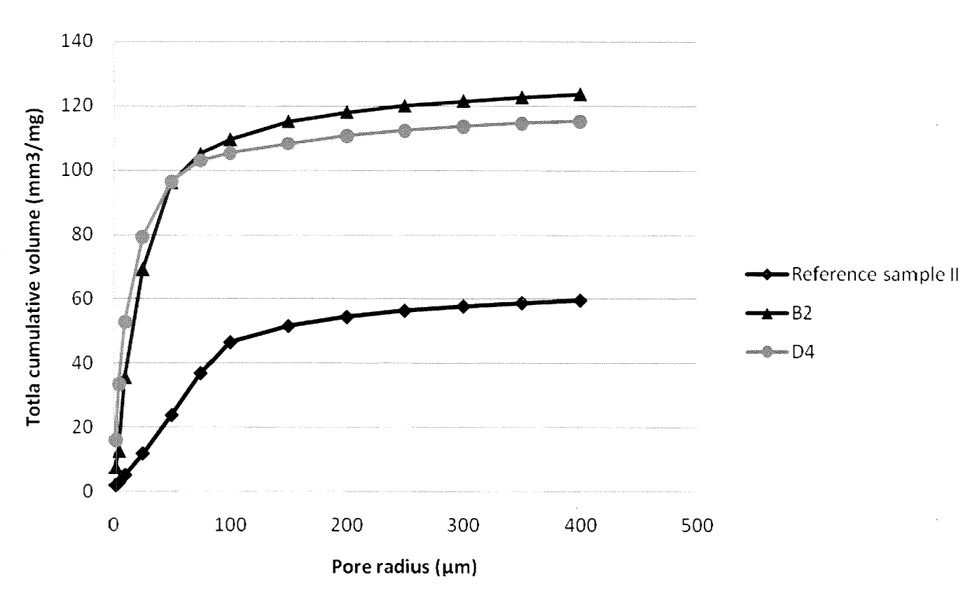

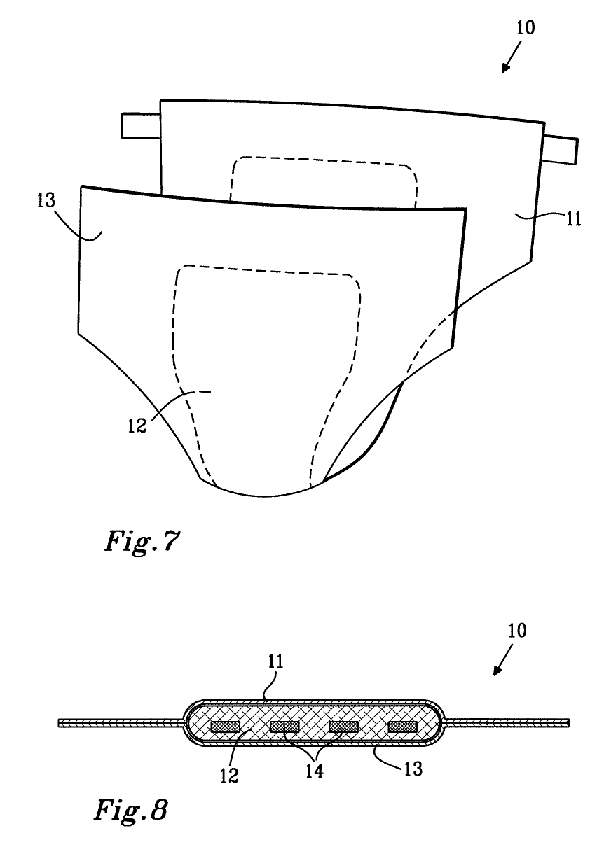

An absorbent article 10 in the form of an open diaper is shown in FIG. 7. The absorbent article 10 typically includes a liquid-permeable topsheet 11, a backsheet 13 and an absorbent body 12 enclosed between the liquid-permeable topsheet 11 and the backsheet 13. The composite material incorporating the absorbent material; i.e. the absorbent porous foam is present in the absorbent body 12.

The liquid permeable topsheet 11 faces the wearer's body during use and is arranged to absorb body liquids such as urine and blood. The material of the topsheet 11 may e.g. be a nonwoven material of spunbond type, a meltblown material, a carded bonded wadding etc.

The backsheet 13 is typically liquid-impermeable, optionally breathable and may e.g. be a plastic (e.g. polyolefin) film, a plastic coated nonwoven or a hydrophobic nonwoven.

The absorbent body 12 acts to receive and contain liquid and other bodily exudates. As such, it may contain the absorbent porous foam; i.e. the composite material, and may contain additional absorbent materials. Examples of commonly occurring absorbent materials are cellulosic fluff pulp, tissue layers, superabsorbent polymers, other types of absorbent foam materials, absorbent nonwoven materials or the like.

The absorbent body 12 may be constructed from several layers, such as a liquid acquisition layer, a storage layer and a distribution layer in order to fulfill the functions which are desired with an absorbent body; i.e. capacity to quickly receive liquid, distribute it within the body and store it.

Since the absorbent porous foam has multifunctional absorption properties with respect to liquid absorption, acquisition, and storage capacity, the composite material may simultaneously fulfill the functions of a liquid acquisition layer, liquid distribution layer and liquid storage layer.

Hence, the absorbent body 12 may include at least one of a liquid acquisition layer, a storage layer and a distribution layer or any combination thereof, and the composite material is present in, or constitutes, at least one of these layer(s).

The layers of the absorbent body 12 are designed to receive a large amount of liquid in a short time and distribute it evenly across the absorbent body. The composite may be present in one or more such layers, and even in all layers. The absorbent body may also fully consist of the composite material.

The size and absorbent capacity of the absorbent body 12 may be varied to be suited for different uses such as for baby diapers, sanitary napkins and incontinence pads.

FIG. 8 is a transverse cross-sectional view of an absorbent article 10, such as the diaper shown in FIG. 7, through the mid-point of the article. It shows a liquid-permeable topsheet 11, a backsheet 13 and an absorbent body 12 enclosed between the liquid-permeable topsheet 11 and the backsheet 13. In the embodiment illustrated in FIG. 8, the absorbent body 12 or at least one layer thereof includes fractions of the composite material housing the absorbent material (porous foam) (shown as 14) mixed with a second absorbent material.

The second absorbent material may be a conventional material used in an absorbent body, e.g. cellulosic fluff pulp, tissue layers, absorbent foam materials, absorbent nonwoven materials or superabsorbent polymers (SAP).

Accordingly, the composite material 14 is cut into smaller fractions or pieces, which are applied in localized areas of the absorbent body. When such fractions are mixed with a second absorbent material, e.g. a material including superabsorbent polymer(s), the spreading and wicking of the liquid within the absorbent body or layer(s) thereof may be improved. This has the advantage that liquid is more efficiently spread within the absorbent body or a layer thereof.

### Preparation of an Absorbent Material According to an Embodiment of the Invention

### EXAMPLE 1

### Oxidation of Cellulosic Pulp

12.0 g (oven dry; o.d.) a bleached never dried softwood sulphate pulp was added to a 1.20 L solution containing 0.1 mM TEMPO (2,2,6,6-tetramethylpiperidin-1-yloxy, free radical) and 1 mM NaBr (sodium bromide). After the pulp addition the suspension was adjusted to pH 10 with 1M NaOH. The reaction was started by adding a certain amount of NaOCl (sodium hypochlorite solution) solution adjusted to pH 10. The amount of NaOCl added was different for the four pulps produced (referred to as A, B, C and D), as described in table 1, to obtain pulps with different content of charged groups; i.e. carboxylate groups. The reaction was carried out at room temperature in a 2 L glass vessel and the suspension was continuously stirred using a magnetic stirrer. To avoid a decrease in pH during the reaction 1M NaOH was added dropwise to maintain the pH between 9.75 and 10.25. The reaction was stopped when no further decrease in pH was observed. The reaction time was longer with a high dosage of NaOCl, with a maximum of 150min at 5 mmol NaOCl/g cellulose pulp.

After the reaction, the pulp was placed in a Büchner funnel with a nylon web (distance between wires: 200 μm, diameter of wires 400 μm) and the liquid was separated from the oxidized pulp. The filtrate was returned once to reduce the loss of fine material. After that it was washed with at least 0.4 L deionized water per gram of oxidized pulp.

TABLE 1
Addition of NaOCl

| Sample | Conc. NaOCl (mmol/g pulp (o.d.)) |
|---|---|
| Pulp A | 1 |
| Pulp B | 2.5 |
| Pulp C | 3.8 |
| Pulp D | 5 |

The oxidation with the TEMPO-radicals facilitates the oxidation of the hydroxyl groups, at carbon 6 in the cellulose chains, to both carbonyl and carboxylate groups. After the oxidation the pulp is referred to as Tempo oxidized pulp (TOP).

### EXAMPLE 2

### Content of Charged Groups

The content of charged carboxylate groups in the pulp samples after the oxidation step was determined by sorption of methylene blue. Approximately 0.05 g (o.d.) TEMPO oxidized pulp was added to a beaker with 100 mL 0.01 HCl. The suspension was stirred for 1 h with a magnetic stirrer. Thereafter, the pulp was washed with a portion of 50 mL 0.01M HCl and two portions of deionized water. To reduce the content of water in the sample it was carefully dewatered. In the next step, the dewatered sample was added to a beaker together with 100 mL of buffer containing methylene blue. The methylene blue buffer contained 0.002M NaH2PO4, 0.0078M Na2HPO4 (buffer adjusted to pH 7.8), 0.4798 g methylene blue, and deionized water to a total volume of 1.00 L.

The sorption was conducted in darkness for 1 hour. After that the reaction liquid and the fibres were separated by filtration. The filtrate was diluted to 125 times its original volume and analyzed on a Hitachi U-3200 spectrophotometer. The absorbance was measured at 664 nm. The fibres were collected on a filter paper and were then washed with 200 mL of 0.01M HCl to desorb the methylene blue from the fibres. After that the fibres were further washed with deionized water, dried in an oven at 105° C. for at least 4 hours, and then the weight of the fibres were measured. The content of charged groups was calculated with the consumption of methylene blue and the fibre weight

As shown in table 2, the content of charged carboxyl groups is increased by the TEMPO oxidation reaction. Bleached softwood paper pulp without any NaOCl treatment is referred to as “Reference pulp I” in table 2. The hydroxyl groups at carbon 6 in the cellulose chain are selectively transformed into charged carboxyl groups.

TABLE 2
Content of charged groups

| | Conc NaOCl | Charged groups |
|---|---|---|
| Sample | (mmol/g pulp (o.d.)) | (mmol/g) |
| Reference pulp I | 0 | 0.07 |
| TOP_A | 1.0 | 0.42 |
| TOP_B | 2.5 | 0.92 |
| TOP_C | 3.8 | 1.02 |
| TOP_D | 5.0 | 1.38 |

### EXAMPLE 3

### Mechanical Treatment of the Oxidized Pulp

The TEMPO oxidized pulps in table 2 were then mechanically treated by homogenization.

5.0 g TOP was suspended with water in a plastic beaker to a solid content of 1%.

The TOP was homogenized by a high shear laboratory batch mixer, such as Ultra-Turrax T 45/N (IKA WERK) speed: 10,000 rpm, rotor diameter: 40 mm, stator diameter: 45 mm. The fibres in the pulp were disintegrated into finer structures

After the mechanical treatment the material changed its form from a hydrophilic pulp to a more gel-like material. This material is referred to as Homogenized tempo oxidized pulp (HTOP). All durations of mechanical treatment in this document are based on samples of 5 g (dry substance).

In table 3, the final content of solids is presented for each of the samples. Samples were collected after 1, 3, 5, 10 and 15 minutes, respectively.

TABLE 3
Solid content (%) of homogenized TEMPO oxidized pulps

| | 1 min | 3 min | 5 min | 10 min | 15 min |
|---|---|---|---|---|---|
| HTOP A | | | 1 | 1 | 1 |
| HTOP B | | | 1 | 1 | 1 |
| HTOP C | 1 | 1 | 1 | ⅔ | 1 |
| HTOP D | 1 | ⅔ | ⅔ | ⅔ | ½ |

During homogenization, the viscosity of the suspension increased. Some of the suspensions (HTOP C and D) became too viscous such that dead zones were created in the sample beaker. To provide a good mixing of the entire volume of these samples, they were diluted with a portion of deionized water to enable further treatment.

During the treatment, liberated fibrils were suspended due to the content of charged carboxylate groups.

HTOP A and B were not collected at 1 min and 3 min because these pulps were not as easy to disintegrate (due to a lower content of charged groups).

### EXAMPLE 4

### Fibre Fractionation

Fractionation between long and short fibres in the HTOP samples of Table 2 was conducted in order to show the relative ease of disintegration of the fibres.

10 g of the HTOP samples of Table 2 having a concentration between 0.5-1%, was added to a beaker.

80 ml of deionized water and 10 ml of 0.1M HCl was subsequently added. The suspension was then gently stirred with a magnetic stirrer for 1 h. The addition of acid protonized the carboxylic acid groups, which facilitated the liberation of the individual fibre fragments into the suspension. Prior to the actual fiber fractionation, the pH was set to 7 by adding 0.5M NaOH dropwise.

The amount of long vs. short fibres was determined by separating the fibre fractions using a Dynamic Drainage Jar, manufactured by Paper Research Materials. The Dyanamic Drainage Jar, manufactured by Paper Research Materials, consists of a vessel with a stirring device, a metallic screen with conical holes (metallic screen 40M was used which is about equivalent to a ordinary quadratic 50 Mesh net) and plastic tube in the bottom to collect the filtrate (no bottom glass cone was used).

The sample was then diluted to a total volume of approximately 500 mL using deionized water. The diluted sample was added to the drainage vessel (bottom tube closed) and stirring was started for 15 s at 1500 rpm (revolutions per minute). After that the stirring speed was adjusted to 750 rpm and the bottom tube was opened so the water and the short fibres could be drained into a beaker. After the drainage, the short fibre fraction and the long fibre fraction were collected and both were diluted to a total weight of each suspension of 500 g. The solid contents of the suspensions were determined isolation of the solid material by filtration followed by weighing after drying at 105° C. for four hours.

In Table 4, the % short fraction is given; i.e. the fraction wherein the microfibrillated cellulose is present. The time in the sample names refers to the mechanical treatment time.

Reference pulp I is bleached softwood paper pulp (no oxidation, no mechanical treatment)

Reference pulp II is bleached softwood paper pulp treated by homogenizing for 15 minutes.

TABLE 4
Short fibre fraction

| | Short fraction |
|---|---|
| Sample | (%) |
| Reference pulp I | 32 |
| Reference pulp II | 41 |
| HTOP A_10 min | 24 |
| HTOP B_10 min | 54 |
| HTOP C_10 min | 81 |
| HTOP D_1 min | 41 |
| HTOP D_3 min | 69 |
| HTOP D_10 min | 77 |
| HTOP D_15 min | 80 |

Table 4 shows how the mechanical disintegration is enhanced by a higher content of carboxylate groups. More material is transferred from the long fraction to the short fraction. Furthermore, the disintegration of pulp into MFC by homogenization is enhanced with longer mechanical treatment.

### EXAMPLE 5

### Freeze-Drying the Microfibrillated Cellulose

The samples of Example 4 were subsequently subjected to freeze-drying by freezing the samples rapidly in a glass beaker with liquid nitrogen. Then, the beakers were placed in a freeze-dryer (Hetosicc CD 2.5 from Heto) at a pressure of 0.3 to 0.5 mbar, and the water was removed by sublimation. The time of drying was 60 hours to ensure that the samples were dry.

The resulting materials were porous foams with slightly different foam characteristics depending on the amount of charged groups and freeze-drying. The material is referred to as Freeze-dried homogenized tempo oxidized pulp (FD-HTOP).

### Characterization of the Absorbent Foam

### EXAMPLE 6

### Determination of Pore Volume Distribution and Total Cumulative Volume

The pore volume distribution for different liquid-permeable covering materials and liquid-transfer materials was determined using the method described in Journal of Colloid and Interface Science 162, 163-170 (1994). The method used is based on measurements of the quantity of liquid which can be released from a porous material (“receding mode”) at a certain pressure, and the result of the measurement is presented in the form of a curve in a chart where the curve illustrates the overall pore volume for a given pore radius.

In the measurements, n-hexadecane (greater than 99 percent, Sigma H-0255) was used as the measuring liquid. Measurement was carried out on circular samples with an area of 15.9 cm². The sample was placed in the chamber and was saturated with the test liquid. Millipore 0.22 μtm cat. no. GSWP 09000 was used as the membrane. In order for it to be possible to record the remaining liquid, the sample was weighed before and immediately after running was completed.

The equilibrium speed, that is to say the speed when the weight change at the selected pore radius has decreased to an insignificant level, was set at 5 mg/min, and the measuring time during which the weight change was recorded was set at 30 seconds.

Measurements were carried out at pressures corresponding to the following pore radii {μm}: 400, 350, 300, 250, 200, 150, 100, 75, 50, 25, 10, 5, and 2 (assuming that the surface tension is 27.7 mN/m of the liquid and that the liquid completely wets the structure).

FIG. 6 shows the total cumulative pore volume, PVr, (index refers to the pore radius, r) of all voids having a corresponding pore radius being less than the actual pore radius, r, represented in the figure. The cumulative pore volume for pores with corresponding pore radii in an interval from a smaller pore radius a to a larger pore radius b may be calculated as follows:

PVa−b=PVb−PVa

The liquid trapped at high capillary pressures e.g. in the walls of the foam are expected to be in voids with small corresponding radius below 2 μm. The larger pores refer to the volume of liquid that may be captured in the voids between the walls of the foam. A foam with large cells and highly absorbent walls is defined by a large cumulative volume below 2 μm, total cumulative pore volume of more than 5 mm3/mg, preferably more than 10 mm3/mg, and also a significant pore volume in voids corresponding walls in the region of 10 μm to 50 μm, pore volume more than 20 mm3/mg, preferably more than 40 mm3/mg. Such a foam is useful as it has larger voids that may give better liquid transportation and smaller voids that have better retention properties.

### EXAMPLE 7

### BET Surface Area

The surface area of the freeze-dried materials of Example 5 was measured by Micromeritics Tristar, an automated gas adsorption analyzer. Samples were first placed in test tubes and pretreated in inert atmosphere for 3 hours at 25° C. in a Micromeritics Smartprep—programmable degas system. After pretreatment the test tubes were placed in the analyzer. Nitrogen gas was used in all experiments. The surface area for the freeze-dried samples of Example 5 was calculated by the BET-method (Table 5).

The freeze dried HTOP_A sample of table 4 (having a lower content of charged groups) did not exhibit the desired foam characteristics, and was less stable in the wet state. In the following, this sample is referred to as Reference sample III.

In Table 5, the following samples were measured:

Reference samples I and II refer to the freeze-dried reference pulp I and II.

Sample B1: absorbent foam comprising 0.92 mmol/g of charged groups; 10 min mechanical treatment

Sample C1: absorbent foam comprising 1.02 mmol/g of charged groups; 10 min mechanical treatment

Sample D1: absorbent foam comprising 1.38 mmol/g of charged groups; 1 min mechanical treatment

Sample D2: absorbent foam comprising 1.38 mmol/g of charged groups; 3 min mechanical treatment

Sample D3: absorbent foam comprising 1.38 mmol/g of charged groups; 10 min mechanical treatment

Sample D4: absorbent foam comprising 1.38 mmol/g of charged groups; 15 min mechanical treatment

TABLE 5
BET surface area

| Sample | BET surface area (m²/g) |
|---|---|
| Reference sample I | 15.9 |
| Reference sample II | 21.7 |
| Reference sample III | 9.6 |
| B1 | 14.9 |
| C1 | 31.4 |
| D1 | 30.2 |
| D2 | 35.7 |
| D3 | 34.6 |
| D4 | 64.9 |

The measurements of the surface area show that the surface area increases with the content of charged groups. Furthermore, the surface area increases with mechanical treatment time. When the content of charged groups is lower, it might be necessary to apply a longer mechanical treatment period, which may be the case with sample B 1.

### EXAMPLE 8

### Scanning Electron Microscopy

Scanning Electron microscopy was used to study the structure of reference sample III and D3 in Example 7. A sample was prepared by first taking out a small sample of freeze-dried homogenized tempo oxidized pulp from a freeze-dried sample. Then the surfaces of the sample were sputtered with an approximately 20 nm thick layer of gold ions with a JEOL JFC-1100E ion sputter. After the coating step, the samples stubs were placed in a JEOL JSM-820 scanning microscope at acceleration voltage of 20 kV. Digital photos of the samples were collected by the JEOL Semafore SA20 slow scan digitalizer and the Semafore 5.1 software.

FIGS. 1a and 1b illustrate the fibre network of sample D3, and reference sample III, respectively. The magnification is 370×, and 350×, respectively, and the markers represent 100 μm.

### EXAMPLE 9

### Stability of the Absorbent Porous Foam

Determination of the Content of Carbonyl Groups with Sodium Chlorite

An oxidation with sodium chlorite was performed to determine the content of carbonyl groups in the pulp. The sodium chlorite oxidizes the carbonyl groups in this slow reaction. The content of carbonyl groups is then calculated by the increase in the content of charged groups compared with a sample not oxidized with sodium chlorite. 0.05 g of pulp sample was added to a mixture of 10 mL of 0.5M CH3COOH, 5 mL of 0.5M NaOH, 0.04 g of NaClO2 and 85 mL of deionized water. The pH of the solution was 4.6. The pulp suspension was stirred during the 24 h reaction time. After the reaction the pulp was washed with 200 mL of deionized water. The content of charged groups was then determined by the method with sorption of methylene blue, see example 2.

Reduction of Carbonyl Groups with Sodium Borohydride

A reduction of the oxidized pulp (TOP_D) was performed to decrease the content of carbonyl groups. 5 g of oxidized pulp was suspended in water (solid content 8%) together with 0.303 g NaBH4 and 0.115 g 0.05 mM NaOH. The suspension was poured into a plastic bag and the plastic bag was put in a water bath (60° C.) for 2 hours. During the reaction, carbonyl groups were reduced to hydroxyl groups. After the reaction time ended the pulp was cooled by dilution with cold water and then the sample was dewatered and washed with deionized water.

The stability of the foam was analyzed by providing samples with different amounts of carbonyl groups.

Sample 1: sample D4 as above.

Sample 2: same as sample D4, but the oxidized pulp was treated with sodium borohydride before the mechanical treatment (to reduce the amount of carbonyl groups).

Sample 3: reference sample 1 mechanically treated for 120 minutes.

All three samples were put in beakers with a large excess of water. Sample 1, containing carbonyl groups in an amount of 0.61 mmol/g of MFC, recovered to its original size and shape after an initial shrinkage during the rapid intake of water. The bonds formed in this sample provide a stable porous foam in the wet state. The size and shape of the sample also recovered after a compression to 20% its height. This indicates that the fibrils of the MFC are held together by these strong bonds. In sample 2, having a content of 0.14 mmol carbonyl groups per g of MFC, the sample returned to a gel-like state after wetting. A compression broke the sample into several pieces. Sample 3 (0.03 mmol carbonyl groups per gram of cellulose) was completely dispersed when the sample was wetted. This indicates that this sample does not have bonds to preserve the fibril network in the presence of water.

In conclusion, the results strongly indicate that the carbonyl groups present in the absorbent porous foam of the present invention create interfibrillar covalent bonds, which are important for the preservation of foam in the wet state. It has previously been proposed to use crosslinking agents to bond microfibrillar material together, but in the absorbent material, crosslinking agents are not required.

### EXAMPLE 10

### Absorption Properties

Absorption experiments were conducted to evaluate the absorption properties of the absorbent foams including a higher content of charged groups (sample D4). Comparative experiments were made for HTOP D samples which had been air dried instead of freeze-dried (15 min mechanical treatment).

The experiments were conducted in deionized water, and 1.0% by weight of NaCl solution, respectively. First the dry weight of the sample was measured. At each measurement, the sample was lowered into a beaker at time zero and was allowed to absorb for 1 minute, 3 minutes, 5 minutes and 10 minutes, respectively. Then the clock was stopped and the sample was taken out of the solution, free water was allowed to drip off and the weight was measured. Thereafter, the sample was put back into the beaker and the clock was started again.

Tests were also performed on sample D4 when the material had been compressed at least 30 times its original height.

The HTOP samples subjected to air-drying were poured out on top of a plastic lid and left to dry at room temperature for several days. The result was a thin film with different amount of fibres present depending on the level of oxidation and mechanical treatment.

The material dried by the air-drying will hereinafter be referred to as air dried homogenized tempo oxidized pulp (AD-HTOP).

In table 6, the absorption liquid is indicated in the parenthesis. The values in the table are given as weight of liquid absorbed per weight of dry sample.

TABLE 6
Absorption properties in g/g

| Sample | 1 min | 3 min | 5 min | 10 min |
|---|---|---|---|---|
| D4 (water) | 158 | 173 | 184 | 182 |
| D4_compressed (water) | 48.3 | 93.7 | 122 | 140 |
| D4 (1% NaCl) | 121 | 148 | 153 | 141 |
| D4_compressed (1% NaCl) | 54.2 | 70.4 | 72.2 | 75.9 |
| AD_HTOP (water) | 3.4 | 5.4 | 6.5 | 8.7 |
| AD_HTOP (1% NaCl) | 2.0 | 2.4 | 2.4 | 2.6 |

The absorption experiments showed big differences in absorption speed and capacity between foam vs. the thin film as obtained by air-drying the HTOP samples.

The air dried film did not absorb much liquid in neither the salt solution nor the water, and after 90 min, no significant increase in absorption was observed. For foam samples (D4) the initial absorption velocity was very fast, because of the open and porous structure of the material. The absorption after 10 minutes was as large as 182 g/g, which is about the same as the theoretical value of absorption calculated as the volume of void in the dried material.

High absorption values were obtained even after 1 minute, 3 minutes, and 5 minutes, respectively. The absorption speed was high even when the material had been compressed. The absorption speed and capacity was high also when using salt solution, but not as high as for deionized water.

### EXAMPLE 11

### Wet Bulk

To evaluate the behaviour under an external load, the wet bulk was measured for two absorbent foams (B2 and D4) when subjected to different external loads. The test liquid used was deionized water. The solid content in the homogenized TEMPO oxidized pulps prior to freeze-drying was 0.6%.

A cylinder with an inner diameter 5 cm having a bottom made of a liquid permeable metallic net screen was used. The net must withstand and be stable at a load of 20 kPa. A thickness meter capable of possessing a load on the sample meanwhile measuring the thickness was also used. A light flat acrylic plate of the same diameter as the inner diameter of the cylinder was placed on top of the metallic net. This acrylic plate is hereinafter referred to as the lid. The weight of the lid should be carefully registered when the lid is still dry. The thickness meter is tared to 0 mm inside the cylinder on top of the lid placed on the metallic net inside the cylinder.

The sample 5 cm in diameter was weighed, and the weight was registered. Thereafter the sample was placed in the cylinder. The lid was placed on top of the sample. The load from the thickness meter and the lid together should be 0.7 kPa. The set-up was left to be stable for 2 minutes. Thereafter the thickness T1 was measured and registered. The dry bulk could be calculated:

Dry bulk=T1[cm]*Area of sample[cm2]/Weight of dry sample[g]

A clean beaker with the inner diameter of 10.4 cm was filled with 80 ml of deionised water. The cylinder with the sample was gently placed in the beaker. Preferably, the beaker is placed around the sample without moving the sample. The sample was allowed to absorb liquid for 10 minutes under the load of only the lid (0.07 kPa). The beaker with liquid was gently withdrawn and the sample was allowed to rest for 2 minutes (no measurement). Then a total load of 0.1 kPa was applied and the system was resting for 2 minutes.

The thickness reading, TW, was thereafter made and registered and the wet bulk could be calculated:

Wet bulk=TW[cm]*Area of sample[cm2]/Weight of dry sample[g]

Loads were applied in sequence according to Table 7. For each new load the set-up was resting for 2 minutes before the reading of the thickness.

If the sample had an area that was not that of a cylinder with diameter 5 cm the applied load was adjusted for the actual sample area. A sample that is not pre-shaped as a layer could be tested if the sample is evenly spread over the metallic screen.

Table 7 illustrates the wet bulk for two foam samples according to embodiments of the invention; i.e. B2 (similar to sample B1 above, but the mechanical treatment time is 15 minutes) and D4, compared to reference sample II (i.e. freeze-dried reference pulp II).

TABLE 7
The wet bulk (cm3/g)

| Load | Reference sample | | |
|---|---|---|---|
| (kPa) | II | B2 | D4 |
| 0.1 | 24.0 | 68.6 | 93.5 |
| 0.7 | 12.5 | 37.0 | 47.5 |
| 1.3 | 9.5 | 26.5 | 37.7 |
| 2.6 | 7.0 | 17.4 | 24.2 |
| 5.2 | 5.2 | 11.0 | 18.0 |
| 7.6 | 4.4 | 8.5 | 15.3 |
| 12.5 | 3.8 | 7.0 | 12.9 |
| 18.8 | 3.5 | 6.1 | 11.4 |

### EXAMPLE 12

### Free Swell Capacity (FSC)

The free swell capacity was measured by the standard test Edana 440.1-99, wherein the step of dripping for 10 minutes has been changed to 2 minutes. The free swell capacity was also measured for 1, and 5 minutes, respectively.

The same samples as used in the wet bulk test were used for these measurements.

TABLE 8
The free swell capacity (g/g)

| Time | Reference sample II | B2 | D4 |
|---|---|---|---|
| 1 min | 24.3 | 63.4 | 48.4 |
| 5 min | 22.1 | 56.7 | 51.0 |
| 30 min | 21.9 | 53.9 | 56.2 |

The results of table 8 are illustrated in FIG. 3.

### EXAMPLE 13

### Centrifuge Retention Capacity (CRC)

The centrifuge retention capacity was measured by the standard test Edana 441.1-99.

The same samples as used in the wet bulk test were used for these measurements.

TABLE 9
The centrifuge retention capacity (g/g)

| Time | Reference sample II | B2 | D4 |
|---|---|---|---|
| 3 min | 4.43 | 8.92 | 12.62 |

The results of table 9 are illustrated in FIG. 4.

### Preparation of a Composite Material

The oxidation and mechanical treatment of the cellulosic pulp were performed as explained in Example 1-4. Thereafter, the gel like microfibrillated cellulose (HTOP) was mixed with different types of cellulosic pulps.

A stirrer from IKA was used to mix the HTOP material with the cellulosic fibers. A fixed amount of HTOP material was used for every sample, such that the amount of fibres determined the ratio of fibres and HTOP material. Stirring was continued until a homogenous suspension was attained.

TABLE 10
The ratio of fibres and HTOP material for the different pulp
types used in the preparation of the composite material.

| | Fiber/HTOP (g/g) | | | | | | | |
|---|---|---|---|---|---|---|---|---|
| Fiber type | 0.18 | 0.33 | 1 | 1.9 | 3 | 4 | 5.7 | 7.3 |
| SKP | x | x | X | | x | | x | |
| CTMP | | x | X | X | x | x | x | x |
| HTCTMP | | x | X | | x | | x | |

Samples of absorbent material only and fibres only were also prepared (the samples with fibres only had the same dry weight as the 4 g fibre/g HTOP samples) for comparison. The suspensions were freeze dried as in example 5 (20 g of wet suspension was used for each sample).

The suspensions which had a solid content between 0.6 - 5.0%, were freeze dried. 20 g suspension was added to 100 mL glass beakers. The samples were freezed with liquid nitrogen and placed in a freeze dryer (Hetosicc CD 2.5 from Heto) until the material was considered dry (about 48 hours). The pressure during freeze drying was about 0.3 mbar and the condenser temperature was at −55° C. After drying, the samples were put in plastic bags and stored at room temperature.

### Characterization of the Composite Material

### EXAMPLE 14

### Scanning Electron Microscopy

In accordance with Example 8, scanning electron microscopy was used to study the structure of the composite material. FIG. 1c illustrates the SEM structure of a composite material comprising CTMP fibres (5.7 g CTMP/g absorbent material).

### EXAMPLE 15

### Wet Bulk

The wet bulk of the composite materials (plus samples containing 0% and 100% fibres) was measured in accordance with example 11, apart from that a saline solution (0.9% NaCl by weight) was used as the test liquid instead of deionized water. The loads were slightly different compared to example 11, due to the sample area and the loads used for the composite materials are presented below.

Load (kPa)

0.1
0.7
1.3
2.6
5.2
7.7
12.9
19.4

The wet bulk of a composite material including various amounts of (i) softwood kraft pulp (SKP), (ii) chemithermomechanical pulp (CTMP), and (iii) high temperature chemithermomechanical pulp (HTCTMP) is illustrated in FIG. 2b. Measurements were also made for absorbent material only, as well as for each of the pulp type only. The dotted lines in FIG. 2b represent the theoretical wet bulk, which would be expected for a composite including each of the pulp type.

### EXAMPLE 16

### Centrifuge Retention Capacity in the Composite Materials

The centrifuge retention capacity test was performed as in example 13. The samples tested were samples of fibres only, absorbent material only and composite materials with various amounts and different types of fibres. The measured CRC values were then used for the calculation of CRC2 (see formula below). CRC2 is the retention capacity of the absorbent material if the improved capacity of the composite material is totally assigned to the absorbent material. This assumption is reasonable due to limitations for fibres to retain a large amount of liquid. In the formula, it can be seen that the contribution to the CRC from fibres only is subtracted, and so is the weight of the fibres. Thereby the CRC2 can be defined as the CRC of the absorbent material when used together with fibres in the composite material of embodiments of the invention.

$$CRC 2 = \frac{{( CRC \times m )}_{composite} - {( CRC \times m )}_{fibres}}{{m}_{composite} - {m}_{fibres}}$$

As illustrated in FIG. 4b, in particular embodiments, a low fibre addition softwood kraft pulp (SKP) is the preferred pulp type, while addition of mechanical pulp (CTMP or HTCTMP) is preferable at high fiber additions. The higher stiffness of the CTMP and HTCTMP pulp fibres seem to be favourable to generate stable networks at high fiber additions. The highest CRC was obtained at high concentration of pulp fibers, which indicates a better preservation of the porous structure (less structure collapses) at high fiber additions.

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. For example, the present invention is not limited to the use of specific type of cellulosic pulp. Furthermore, the present invention is not limited to a specific method to impart the plurality of charged groups onto the microfibrillated cellulose, but any suitable method may be used.

1. An absorbent article comprising a freeze dried composite material; said freeze dried composite material comprising cellulosic pulp and an absorbent material, wherein said absorbent material comprises microfibrillated cellulose in the form of an absorbent porous foam; said microfibrillated cellulose (MFC) having a content of carboxylate groups of from 0.5 to 2.2 mmol/g of MFC.

2. The absorbent article according to claim 1, wherein said composite material comprises at least 5% by weight of absorbent material.

3. The absorbent article according to claim 2, wherein said composite material comprises of from 10 to 50% by weight of absorbent material.

4. The absorbent article according to claim 1, wherein said pulp is chemithermomechanical pulp (CTMP).

5. The absorbent article according to claim 1, wherein the content of carboxylate groups in said microfibrillated cellulose is from 0.8 to 1.8 mmol/g of MFC.

6. The absorbent article according to claim 1, wherein the content of carbonyl groups in said microfibrillated cellulose is at least 0.2 mmol/g of MFC.

7. The absorbent article according to claim 1, wherein said absorbent material has a BET surface area of at least 24 m²/g.

8. The absorbent article according to claim 1, wherein said absorbent material has a wet bulk of at least 10 cm³/g at 5 kPa.

9. The absorbent article according to claim 1, wherein said absorbent material has a free swell capacity (FSC) value of at least 45 g/g.

10. The absorbent article according to claim 1, wherein said absorbent material has a retention capacity as determined by a Centrifuge Retention Capacity (CRC) Test of at least 8 g/g.

11. The absorbent article according to claim 1, wherein said composite material is obtainable by:
(a) oxidizing a first cellulosic pulp to obtain a content of carboxylate groups of from 0.5 to 2.2 mmol/g of pulp;
(b) disintegrating said first cellulosic pulp into microfibrillated cellulose;
(c) mixing the microfibrillated cellulose of step b) with a second cellulosic pulp; and
(d) freeze-drying said mixture of microfibrillated cellulose and second cellulosic pulp.

12. The absorbent article according to claim 11, wherein said microfibrillated cellulose and said second cellulosic pulp of step c) are mixed in a wet state.

13. The absorbent article according to claim 10, wherein said second cellulosic pulp is chemithermomechanical pulp.

14. The absorbent article according to claim 12, wherein said oxidation step (a) is performed in the presence of 2,2,6,6-tetramethylpiperidine-1-oxyl (TEMPO).

15. The absorbent article according to claim 1 comprising a liquid permeable topsheet, a backsheet and an absorbent body enclosed between said liquid-permeable topsheet and said backsheet, wherein said composite material is present in said absorbent body.

16. The absorbent article according to claim 10, wherein said absorbent body or at least one layer thereof comprises fractions of said composite material mixed with a second absorbent material.

17. An absorbent structure comprising a freeze dried composite material comprising cellulosic pulp and an absorbent material, wherein said absorbent material comprises microfibrillated cellulose in the form of an absorbent porous foam; said microfibrillated cellulose (MFC) having a content of carboxylate groups of from 0.5 to 2.2 mmol/g of MFC.

18. The absorbent article according to claim 1, wherein the content of carbonyl groups in said microfibrillated cellulose is at least 0.5 mmol/g of MFC.

19. The absorbent article according to claim 1, wherein said absorbent material has a BET surface area of at least 30 m²/g.

20. The absorbent article according to claim 1, wherein said absorbent material has a wet bulk of at least 15 cm³/g at 5 kPa.

21. The absorbent article according to claim 1, wherein said absorbent material has a retention capacity as determined by a Centrifuge Retention Capacity (CRC) Test of at least 12 g/g.
